(19) United States
(12) Patent Application Publication
(10) Pub. No.: US 2020/0029902 A1
(43) Pub. Date: Jan. 30, 2020

(54) BODY-WEARABLE MEDICAL DEVICE

(71) Applicant: Roche Diabetes Care, Inc., Indianapolis, IN (US)

(72) Inventors: Oliver Kube, Mannheim (DE); Helmut Walter, Mannheim (DE); Alexander Poggenwisch, Mannheim (DE)

(21) Appl. No.: 16/589,966

(22) Filed: Oct. 1, 2019

Related U.S. Application Data
(63) Continuation of application No. PCT/EP2018/058566, filed on Apr. 4, 2018.

(30) Foreign Application Priority Data
Apr. 4, 2017 (EP) 17 164 839.7

(51) Int. Cl. A61B 5/00; A61B 5/145

(52) U.S. Cl. CPC A61B 5/6833; A61B 5/14542; A61M 5/1723; A61B 5/14546; A61B 5/68335; A61B 5/14532

(57) ABSTRACT

The disclosure concerns a body-wearable medical device, such as an analyte monitoring system or a patch-mounted pump. The device has a self-adhering flexible electronics patch which adheres to the skin of a user and is deformable to follow the contour of the skin. In order to provide a flexible non-body configuration, the electronics patch includes flexible printed circuitry which is applied directly on a foil substrate. A user interface is configured for a user to control the device.

### RELATED APPLICATIONS

This application is a continuation of PCT/EP2018/058566, filed Apr. 4, 2018, which claims priority to EP 17 164 839.7, filed Apr. 4, 2017, the entire disclosures of each of which are hereby incorporated herein by reference.

### BACKGROUND

This disclosure relates to a body-wearable medical device, such as an analyte monitoring system or a patch-mounted pump.

Such systems are available for monitoring of certain analytes or agents, specifically glucose or lactate in body fluids like blood or interstitial fluid by readings of an implanted sensor, specifically an electrochemical sensor. The subcutaneously implanted sensor remains in the interstitial tissue over an extended period of time even up to several weeks. Then, the in vivo detected measurement signals may be indicative of an analyte, e.g., glucose in the blood of the subject. The monitoring may be a nearly real-time continuous or quasi continuous or periodic approach for frequently providing/updating analyte values without sample handling or similar user interaction.

In present practice, continuous glucose monitoring (CGM)-systems include a so-called bodymount as a patch which has a rigid housing portion or stiff mounting platform on which the electronics unit is mounted and galvanically coupled to the sensor. As the human body is relatively soft and flexible, the rigid housing or platform in connection with the sensor cannot follow the deflections and elongations, thereby resulting in shearing forces which lead to early detachment of the bodymount from the skin. Furthermore, the platform on the body has only reduced breathability, such that humidity accumulates therebelow, which also undesirably reduces the possible wearing time. As a further problem, the user may need a remote control for actuating the device.

WO 2016/187536 A1 describes an ultra-thin wearable sensing device which includes a sensor tag IC that enables the device to communicate wirelessly to a reading device. The wearable sensing device includes one or more sensors connected to the sensor tag IC that sense characteristics of the person, animal or object that the sensing device comes in contact with. The sensed characteristics can include biological signals (e.g., ECG, EMG, and EEG), temperature, galvanic skin response (GSR), heat flux and chemicals or fluids released by the skin. The reading device can display the information to the user and/or transmit the sensor data to a remote location for further processing. A doctor can review the data or have the data further analyzed and use this data or information to assist with treatment.

WO 2016/090189 A1 describes a non-invasive epidermal electrochemical sensor device which includes an adhesive membrane; a flexible or stretchable substrate disposed over the adhesive membrane; and an anodic electrode assembly disposed over the flexible or stretchable substrate including an iontophoretic electrode. The device includes a cathodic electrode assembly disposed adjacent to the anodic electrode assembly over the flexible or stretchable substrate and includes an iontophoretic electrode. Either the cathodic electrode assembly or the anodic electrode assembly also includes a sensing electrode that includes a working electrode and at least one of a counter electrode or a reference electrode. The iontophoretic electrode in either the anodic electrode assembly or the cathodic electrode assembly that includes the sensing electrode is disposed on the substrate to at least partially encompass the working electrode and the at least one of the counter electrode or the reference electrode. The device includes an electrode interface assembly including independent electrically conductive contacts.

U. S. Publication No. 2008/161656 A1 describes a device, system, and method for delivering a device such as a sensor or fluid transport structure or a fluid transport structure sensor combination into, for example, mammalian skin and receiving, analyzing, and displaying signals from the device such as a sensor. A system includes a reusable sensor assembly including a transmitter, microcontroller, and housing plus disposable sensor assembly including a housing having an opening for receiving both the distal end of a biosensor, a sensor insertion guidance structure, and a transmission apparatus for transmitting signals received from the sensor to a reusable sensor assembly for transmission to an external electronic monitoring unit.

U. S. Publication No. 2014/276167 A1 describes a wearable patch and method for automatically monitoring, screening, and/or reporting events related to one or more health conditions (e.g., sleeping or breathing disorders, physical activity, arrhythmias) of a subject.

WO 2013/136181 A2 describes a pump assembly mounted to or supported by a dressing for reduced pressure wound therapy. The dressing can have visual pressure, saturation, and/or temperature sensors to provide a visual indication of the level of pressure, saturation, and/or temperature within the dressing. Additionally, the pump assembly can have a pressure sensor in communication with the flow pathway through the pump, and at least one switch or button supported by the housing, the at least one switch or button being accessible to a user and being in communication with the controller. The pump assembly can have a controller supported within or by the housing, the controller being configured to control an operation of the pump. The pump can be configured to be sterilized following the assembly of the pump such that all of the components of the pump have been sterilized.

WO 2017/003857 A1 describes a flexible, body-mountable analyte sensing device which includes a flexible substrate configured for mounting to skin of a living body. The sensing device additionally includes a sensor probe attached to the flexible substrate and configured to penetrate the skin such that a sensor disposed on the end of the sensor probe can be exposed to an analyte in interstitial fluid. The sensor could be an electrochemical sensor that includes two or more electrodes disposed at the end of the sensor probe and configured to electrochemically detect the analyte. The sensing device is configured to display detected concentrations or other information about the analyte in the interstitial fluid. The flexible substrate of the sensing device is configured to be adhered or otherwise mounted to the skin in a manner that minimally impacts activities of the living body.

U.S. Publication No. 2016/310049 A1 describes techniques for measuring ion related metrics at a user's skin surface are disclosed. In one aspect, a method for operating a wearable device may involve determining, based on output of one or more ion selective field effect transistor sensors, various physiological conditions such as a state of hydration, a state of skin health, or the cleanliness of the wearable device or an associated garment.

### SUMMARY

This disclosure further improves the known systems and provides a design which allows for long-term wear capability and improved user convenience.

This disclosure is based on the idea of providing a comfortable self-adhering flexible electronics patch with integrated electronic interfaces or actuators. As used herein the term “patch” refers to at least one arbitrary shaped fastening element which is configured to be attached directly to the skin of the user, i.e., without using additional or further fastening elements. As used herein, the term “self-adhering” refers to the patch comprising at least one attachment side, for example a bottom side, adapted to attach and/or mount the patch to the skin, wherein the attachment side comprises at least one adhesive and/or is coated with at least one adhesive coating. As used herein, the term “electronics patch” refers to a patch which comprises at least one electronic element. As used herein, the term “flexible electronics patch” refers to the fact that the electronics patch has flexible properties such that the electronics patch is bendable and/or stretchable to follow the contour of the skin. The patch may have a stretchability of at least 20% in at least two directions, preferably in all directions. As used herein “stretchability of at least 20%” refers to that a patch having a length of, for example, 10 cm (centimeters) can be stretched to a length of at least 12 cm (centimeters). Accordingly, it is proposed that the electronics patch includes a flexible printed circuitry or circuits which are applied directly on a foil substrate, and that a user interface is integrated with the patch for allowing the user to directly control the device. As used herein, the term “the electronics patch includes a flexible printed circuitry or circuits” refers to that at least one flexible printed circuitry is part of the patch and/or is integrated within or into the patch, in particular is integrated within at least one substrate of the patch and/or on at least one substrate of the patch and/or is integrated within at least one layer of the patch, and/or that the flexible printed circuitry is embedded within the patch and/or that the flexible printed circuitry is incorporated in the patch. The patch comprises the foil substrate having the flexible printed circuitry printed thereon. Specifically, the at least one flexible printed circuitry may be integrated and/or incorporated and/or embedded in the patch such that the patch itself is arranged and/or configured as an electronic unit. Thus, the at least one flexible printed circuitry may be comprised by the patch itself, without the need of an additional and/or separate element adapted to store or house the flexible printed circuitry such as a housing or base unit or something similar. Thus, the flexible patch can avoid the disadvantages of a rigid platform and is bendable and/or stretchable to follow the contour of the skin. At the same time, the integrated interface allows for user interaction without the need to provide actuators in a stiff housing. Thereby, the overall operating cycle can be prolonged and the user convenience can be significantly improved.

In this context, a further improvement provides that the user interface is an integrated part of the self-adhering flexible electronics patch. It is further preferred that the user interface is directly applied to the foil substrate. As used herein, the term “the user interface is an integrated part of the self-adhering flexible electronics patch” refers to that the user interface is part of the patch and/or is comprised within or into the patch, in particular, is integrated within at least one substrate of the patch and/or on at least one substrate of the patch and/or is integrated within at least one layer of the patch, and/or that the user interface is embedded within the patch and/or that the user interface is incorporated in the patch. For example, the patch may comprise the foil substrate having the user interface printed thereon. Thus, the user interface may be comprised by the patch itself, without the need of an additional and/or separate element adapted to store or house the user interface such as a housing or base unit or something similar.

In an advantageous configuration, the user interface comprises at least one switch, wherein the switch is configured to operate a component on the electronics patch, such that a direct user interaction is possible without remote control.

For further improved integration, the switch comprises printed conducting elements applied on the foil substrate.

Preferably, the switch is one of manually operable by the user or automatically operable in dependence of a predefined switching condition.

In this connection, it is also advantageous to use the switch for at least one function of the group comprising power-on/off, delivering bolus doses, and emergency shutdown.

Advantageously, the switch can be configured to power a display component of the system on or off.

In combination with a separate pump system, the switch can be configured to interact with the functionalities of the pump system. For instance, the switch may be used as a bolus button for delivering a bolus. In such an embodiment, the sensor data indicative of a glucose level may be used to determine a corresponding bolus and the determined bolus may be released by the pump when the switch is pressed for example by communicating a corresponding signal to the pump.

Additionally or alternatively, the switch may be configured to provide for an emergency shutdown of a pump. Such a situation can arise when the sensor indicates a glucose level that tends to hypoglycemia, a situation in which the basal insulin delivery needs immediate suspension.

Specifically in connection with a patch pump, which is worn on the body, a flexible switch provided on a flexible printed circuitry is advantageous for manual triggering of bolus doses of insulin. This allows a small-sized implementation for direct user interaction.

In a further advantageous embodiment, the user interface comprises a display component operable for displaying information related to the operation of the device, in particular information related to at least one of device status, measuring results, user guidance, and warnings. Thus, a user information or interaction is possible without external devices in a rigid housing.

In a simplified embodiment, the user interface comprises at least one single LED or an array of LEDs as a display component operable for displaying information related to the use of the device.

A more sophisticated approach provides that the display component is formed as a flexible screen, in particular a flexible OLED screen, and the screen is embedded on the foil substrate or is mounted on the body as a separate flexible patch and communicates with the self-adhering flexible electronics patch over a distance. In the latter case, the sensor patch may be worn on a non-visible body area, whereas the display patch is visibly attached to the body. Then, in combination with a user-activated switching arrangement on the electronics patch, the device can be operated independently of an external remote control.

In this connection it is further advantageous when a data connection between the flexible printed circuitry and the display component is provided by conductive textiles. This allows having the display always visible on top of the clothing.

In order to easily adapt to a varying skin contour, the foil substrate of the flexible printed circuits should have a thickness of less than 1 mm, preferably 10-250 microns and advantageously 50-100 microns, more preferably 60-90 microns and most preferably 70-80 microns. Depending on the stability of the foil, a thickness in the range of 10 to 50 microns might also be feasible.

A further improvement provides that foil substrate is stretchable in at least one direction by more of 20% of its initial length. In an embodiment the foil substrate may be stretchable in at least two directions by more than 20%. In an embodiment the foil substrate is stretchable in all directions by more than 20%. As used herein the term “more than 20%” in an embodiment means that a foil substrate having a length of for example 10 cm (centimeters) can be stretched along its length to at least 12 cm (centimeters). A stretchability in the range of 20% is similar to that of the skin and thus provides an optimized and long-lasting wear comfort.

The electronics patch may comprise at least one deformable electronics element and/or at least one rigid or semi-rigid electronics element. For example, the electronics patch may comprise the at least one flexible printed circuitry including at least one electronic element selected from the group consisting of: at least one conductive path, at least one resistor, at least one capacitor, and at least one battery, wherein the electronic elements may be deformable components. For example, the electronics patch may comprise rigid or semi-rigid components such as one or more of at least one integrated circuit chip, at least one processor, at least one storage medium, at least one antenna, and at least one battery. As used herein, the term “comprises at least one deformable electronics element and/or at least one rigid or semi-rigid electronics element” refers to that the deformable electronics element and/or the rigid or semi-rigid electronics element is part of the patch and/or is integrated within or into the patch, in particular is integrated within at least one substrate of the patch and/or on at least one substrate of the patch and/or is integrated within at least one layer of the patch, and/or that the deformable electronics element and/or the rigid or semi-rigid electronics element is embedded within the patch and/or that the deformable electronics element and/or the rigid or semi-rigid electronics element is incorporated in the patch. For example, the patch may comprise the insulating foil substrate having the deformable electronics element and/or the rigid or semi-rigid electronics element printed thereon, in particular directly. Specifically, the deformable electronics element and/or the rigid or semi-rigid electronics element may be integrated and/or incorporated and/or embedded in the patch such that the patch itself is arranged and/or configured as electronic unit. Thus, the deformable electronics element and/or the rigid or semi-rigid electronics element may be comprised by the patch itself, without the need of an additional and/or separate element adapted to store or house the deformable electronics element and/or the rigid or semi-rigid electronics element such as a housing or base unit or something similar.

In a particular embodiment, the flexible printed circuitry includes at least one of conductive paths, resistors, capacitors and batteries as deformable components.

Another possibility provides that the flexible electronics patch comprises at least one of integrated circuit chips, processors, storage media, antennas and batteries as rigid or semi-rigid components which are distributed such that the electronics patch overall remains deformable to adapt its shape to a varying contour of the skin during use.

Advantageously, the flexible electronics patch comprises a printed battery which consists of functional materials, e.g., a zinc manganese dioxide system, printed on a flexible substrate. In order to provide a large capacity, the printed battery should cover a large area or even the whole patch.

In this context, it is also advantageous when the flexible printed circuitry comprises an antenna for a wireless connection to a remote device, and when the antenna is arranged such that it is not shielded by the printed battery (which may include a metallic foil) in a direction away from the user's body. In specific configurations, multiple antennas may be used above and below the printed battery, or on the side thereof.

In a particular useful embodiment, the analyte monitoring system is formed as a continuous glucose monitoring system comprising a skin-implantable glucose sensor.

For a closed loop operation, it is also preferable that the patch-mounted pump is provided to deliver doses of a medical agent such as insulin to the body of the user.

In a further aspect a method for controlling at least one body-wearable medical device according to any one of the embodiments as described above or described in detail below is proposed. The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlapping fashion. The method may comprise further method steps which are not listed. The method comprises the following steps:

i) adhering a self-adhering flexible electronics patch to the skin of a user, wherein the self-adhering flexible electronics patch is deformable to follow the contour of the skin, wherein the electronics patch includes a flexible printed circuitry which is applied directly on a foil substrate;
ii) controlling the device by the user by using a user interface, wherein the user interface is an integrated part of the self-adhering flexible electronics patch.

With respect to embodiments and definition of the method reference is made to the description of the body-wearable medical device above and as described in further detail below.

Summarizing and without excluding further possible embodiments, the following embodiments may be envisaged:

### Embodiment 1

Body-wearable medical device, such as an analyte monitoring system or a patch-mounted pump, comprising a self-adhering flexible electronics patch which adheres to the skin of a user and is deformable to follow the contour of the skin, wherein the electronics patch includes a flexible printed circuitry which is applied directly on a foil substrate, and wherein a user interface is configured for allowing the user to control the device.

### Embodiment 2

The device according to embodiment 1, wherein the user interface comprises at least one switch, the switch being configured to operate a component on the electronics patch.

### Embodiment 3

The device according to embodiment 2, wherein the switch comprises printed conducting elements applied on the foil substrate.

### Embodiment 4

The device according to embodiment 2 or 3, wherein the switch is one of manually operable by the user or automatically operable in dependence of a predefined switching condition.

### Embodiment 5

The device according to any of embodiments 2 to 4, wherein the switch is used for at least one of the group comprising power-on/off, delivering bolus doses, emergency shutdown.

### Embodiment 6

The device according to any of embodiments 1 to 5, wherein the user interface comprises a display component operable for displaying information related to the operation of the device, in particular information related to at least one of device status, measuring results, user guidance, and warnings.

### Embodiment 7

The device of embodiment 6, wherein the user interface comprises at least one single LED or an array of LEDs as a display component operable for displaying information related to the use of the device.

### Embodiment 8

The device of embodiment 6 or 7, wherein the display component is formed as a flexible screen, in particular a flexible OLED screen, and the screen is embedded on the foil substrate or is mounted on the body as a separate flexible patch communicating with the self-adhering flexible electronics patch.

### Embodiment 9

The device according to any of embodiments 1 to 8, wherein a data connection between the flexible printed circuitry and the user interface is provided by conductive textiles.

### Embodiment 10

The device according to any of embodiments 1 to 9, wherein the foil substrate has a thickness of less than 1 mm, preferably 10-250 microns, more preferably 50-100 microns and most preferably 70-80 microns.

### Embodiment 11

The device according to any of embodiments 1 to 10, wherein the foil substrate is stretchable in at least one direction by more of 20% of its initial length.

### Embodiment 12

The device according to any of embodiments 1 to 11, wherein the flexible printed circuitry includes at least one of conductive paths, resistors, capacitors and batteries as deformable components.

### Embodiment 13

The device according to any of embodiments 1 to 12, wherein the electronics patch comprises a printed battery which consists of functional material printed on a flexible substrate.

### Embodiment 14

The device of embodiment 13, wherein the flexible printed circuitry comprises an antenna for a wireless connection to a remote device, and wherein the antenna is arranged such that it is not shielded by the printed battery in a direction away from the user's body.

### Embodiment 15

The device according to any of embodiments 1 to 14, wherein the analyte monitoring system is formed as a continuous glucose monitoring system comprising a skin-implantable glucose sensor which is at least partially insertable into the skin or fully implantable under the skin.

### Embodiment 16

A method for controlling at least one body-wearable medical device according to any one of the preceding embodiments, wherein the method comprises the following steps:

i) adhering a self-adhering flexible electronics patch to the skin of a user, wherein the self-adhering flexible electronics patch is deformable to follow the contour of the skin, wherein the electronics patch includes a flexible printed circuitry which is applied directly on a foil substrate;
ii) controlling the device by the user by using a user interface, wherein the user interface is an integrated part of the self-adhering flexible electronics patch.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is a 3D-expanded exploded view of a body-wearable glucose monitoring system including a flexible electronics patch;

FIG. 2 shows another embodiment in a view similar to FIG. 1; and

FIG. 3 shows a body-mounted glucose monitoring system in connection with a handheld data acquisition device.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

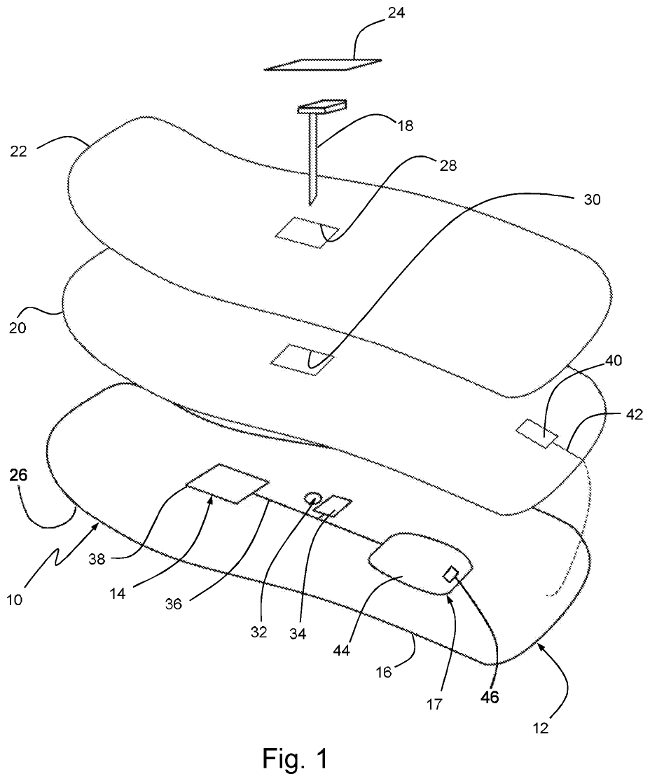

Referring to FIG. 1, a body-wearable medical sensor system 10 for continuous glucose monitoring (CGM) comprises a flexible electronics patch 12 which adheres to the skin of a user and includes a flexible printed circuitry (FPC) 14 which is applied directly on a flexible foil substrate 16, e.g., on a thin polymer film, such that the patch 12 is bendable and/or stretchable to follow the contour of the skin.

As will be detailed further below, a user interface 17 is configured for allowing the user to control the device 10. The user interface 17 may be part of the FCP 14 or may be a separate body-wearable unit connected to the FCP 14. In this connection, allowing the user to control the device 10 means that functional components are provided on-body such that the user is able to directly interact with the device 10, e.g., by reading information or influencing a state of the device without remote control.

In certain embodiments, the system 10 further includes an electrochemical needle sensor 18 which can be partially inserted into the skin, a flexible printed battery 20 (soft battery), a top film 22 as a protective upper cover and a cover film 24 for the sensor 18. In the prefabricated state prior to skin mounting, the foil substrate 16, printed battery 20 and top cover 22 are laminated on another to form a layered flexible assembly which has an adhesive 26 on the underside to attach the patch 12 to the user's skin. Then, the distal part of the needle sensor 18 can be inserted into the skin through openings 28, 30, 32 of the layered assembly by means of an inserter aid (not shown), such that the proximal sensor part contacts a connector 34 of the FPC 14.

The FPC 14 carries flexible printed conducting pathways 36, capacitors, resistors and eventually rigid or semi-rigid electronic components 38, which are all directly mounted on the foil substrate 16. Further rigid elements may include the insertion interface for the sensor 8, which surrounds the insertion opening 32 at least partly, and contact elements such as connectors, printed carbon pills or conductive rubber for the sensor electronic connection. The more rigid components are distributed such that the FPC 14 overall remains deformable to adapt its shape to a varying contour of the skin during use. It may also be conceivable that even processors, antennas for communication and storage media are integrated as flexible components, which would lead to a fully flexible FPC.

In order to maintain sufficient flexibility, the foil substrate has a thickness in the range of 10-250 microns. Preferably, polyimide or polyester films may be used. For following a skin contour under various conditions, it is also advantageous when the foil substrate 16 is stretchable in at least one direction by more of 20% of its initial length. In case of additional stacked layers like printed battery 20 and top film 22, an overall thickness of less than 2 mm, preferably less than 1 mm should be aimed.

The printed battery 20 consists of functional electrode layers and electrolyte materials, e.g., a zinc manganese dioxide system, printed on a flexible foil substrate. An antenna 40 for wireless data transmission is arranged on top of the printed battery 20 such that it is not shielded by the metallic electrode layers. Then, a galvanic connection 42 to the FPC 14 is guided over the rim of the battery substrate. In specific configurations, multiple antennas may be used above and below the printed battery 20, or on the side thereof.

As outlined in FIG. 1, the user interface 17 may comprise a display component 44 (display) which displays information related to the operation of the device 10. Such information may be related to the device status, measuring results, user guidance, warnings etc. The display component 44 may be readable through transparent or cut-out sections in the battery 20 and cover foil 22. Purposively, the display component 44 is formed as a flexible OLED screen, and the screen is embedded on the foil substrate 16.

The user interface 17 may also comprise at least one switch 46 which operates an electronic component of the FPC 14. The switch 46 can be realized by printed conducting elements applied on the foil substrate 16 and operable by manual pressure through the cover foil 22, which may be marked appropriately. Such a switch 46 may be used for power-on/off or emergency shutdown, or other user-initiated functions like delivering bolus doses to the body of the user by means of an insulin delivering patch pump (not shown). It is also conceivable that an integrated switch on the FPC 14 is automatically operable in dependence of a predefined switching condition, e.g., a reading obtained by a sensor.

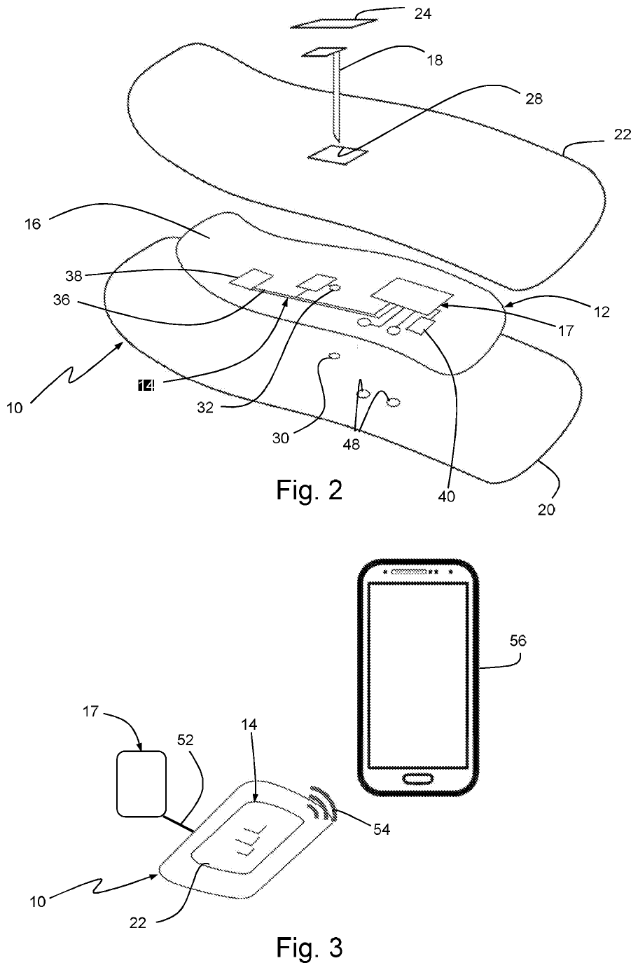

FIG. 2 shows a further embodiment in which the same numerals have been used for same or similar elements as described above. In this embodiment, the flexible printed battery 20 is arranged below the flexible printed circuits or circuitry 14. Consequently, the underside of the battery foil substrate is provided with the adhesive layer for adhering to the skin. Furthermore, the antenna 40 remains on the FPC 14, as it is not shielded by the printed battery 20 in the direction away from the user's body. The battery contact points 48 are through-connected to connection points on the FPC 14 for direct power supply.

FIG. 3 illustrates an embodiment of a body-wearable CGM system 10 in an assembled state mounted on a skin area 50. A data connection 52 between the flexible printed circuitry 14 and a distant interface or display component 17 is provided preferably by conductive textiles. This allows to have the display 17 continuously visible on top of the clothing. Furthermore, a wireless connection 54 can be established via the integrated antenna 40 to a remote handheld data acquisition device 56, which can be provided as a smartphone equipped with an adapted software in the form of an app.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A body-wearable medical device, comprising:
a self-adhering flexible electronics patch configured to adhere to and follow the contour of the skin of a user;
the electronics patch having flexible printed circuitry on a foil substrate, the foil substrate being stretchable in at least one direction by more than 20%; and
a user interface formed as an integrated part of the self-adhering flexible electronics patch, the user interface configured to allow the user to control the device.

2. The device according to claim 1, wherein the user interface has a switch configured to operate a component on the electronics patch.

3. The device according to claim 2, wherein the switch comprises printed conducting elements applied on the foil substrate.

4. The device according to claim 2, wherein the switch is one of manually operable by the user or automatically operable based upon a predefined switching condition.

5. The device according to claim 2, wherein the switch controls at least one of power-on/off, delivering bolus doses and emergency shutdown.

6. The device according to claim 1, wherein the user interface comprises a display operable for displaying information related to at least one of device status, measuring results, user guidance, and warnings.

7. The device according to claim 6, wherein the display has at least one single LED or an array of LEDs operable for displaying information.

8. The device according to claim 6, wherein the display is formed as a flexible screen embedded on the foil substrate or mountable on the body as a separate flexible patch communicating with the self-adhering flexible electronics patch.

9. The device according to claim 8, wherein the display comprises a flexible OLED screen.

10. The device according to claim 1, further comprising conductive textiles that provide a data connection between the flexible printed circuitry and the user interface.

11. The device according claim 1, wherein the foil substrate has a thickness of less than 1 mm.

12. The device according claim 11, wherein the foil substrate has a thickness of 10-250 microns.

13. The device according claim 11, wherein the foil substrate has a thickness of 70-80 microns.

14. The device according to claim 1, wherein the flexible printed circuitry includes at least one of conductive paths, resistors, capacitors and batteries as deformable components.

15. The device according to claim 1, wherein the electronics patch comprises a printed battery formed of functional material printed on a flexible substrate.

16. The device according to claim 15, wherein the flexible printed circuitry comprises an antenna configured for a wireless connection to a remote device, and wherein the antenna is not shielded by the printed battery in a direction away from the user's body.

17. The device according to claim 1, comprising a continuous glucose monitoring system having a skin-implantable glucose sensor which is partially or fully insertable into the skin.

18. A method for controlling at least one body-wearable medical device, comprising:
providing an electronics patch having flexible printed circuitry on a foil substrate;
adhering the patch to the skin of a user wherein the patch follows the contour of the user's skin; and
using an interface integrated into the patch to control device.

19. The method of claim 18, further comprising stretching the foil substrate in at least one direction by more than 20%.

20. The method of claim 18, wherein the step of using an interface comprises powering the device on/off, delivering a bolus dose, or emergency shutdown.
